(19) United States
(12) Patent Application Publication
(10) Pub. No.: US 2009/0018385 A1
(43) Pub. Date: Jan. 15, 2009

(54) URETHRA PRESSURE CONTROL VALVE TO CONTROL INCONTINENCE

(76) Inventor: Antoine Trubiano, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/775,259

(22) Filed: Jul. 10, 2007

(51) Int. Cl. A61F 2/02

(52) U.S. Cl. 600/30

(57) ABSTRACT

A surgically implantable urethra pressure control adjustable valve for male and female patients is described. The valve comprises a clamp positionable about a urethra in a patient's body. The clamp has a circumferential wall having a first and a second opening. The openings are oppositely spaced apart at predetermined locations to provide for the passage of the urethra through the circumferential wall. A pressure abutment element is provided inside the circumferential wall and disposed between an inner wall surface of the circumferential wall and the urethra and actuable to apply a contained control pressure against the urethra to close the urethra by pinching same against a diametrically opposed immovable abutment element formed in the circumferential wall and projecting inwardly therein. The contained controlled pressure is adjustable and selected so that liquid pressure from a bladder associated with the urethra and under the influence of muscle control will cause the urethra to open against the contained controlled pressure to discharge liquid from the bladder and to automatically close once the pressure from the bladder is discontinued by muscle control.

### TECHNICAL FIELD

The present invention relates to a urethra pressure control adjustable valve which is implantable in a patient to cause the urethra to open and close to discharge liquid from the bladder by sphincter muscle control.

### BACKGROUND ART

Urinary incontinence is defined as the accidental leakage of urine through the urethra. Prostate problems and post radical prostatectomy urinary incontinence greatly affects a matters quality of life. The National Institute of Diabetes and Digestive and Kidney Diseases (NIDDK) has reported that urinary incontinence is a medical problem and that there are four forms of urinary incontinence. These are (1) temporary and reversible incontinence related to urinary track infection, constipation or delirium; (2) stress incontinence caused by weak pelvic and sphincter muscles; (3) urgent continence caused by damaged or iritatable nerves; and (4) overflow incontinence that results when an individual is unable to empty the bladder.

The urinary system, to do its job, muscles and nerves must work together to hold urine in the bladder and then release it at the right moment. A person develops the sphincter muscle control as a normal phenomenon associated with nerve signals. These muscles cause the bladder to squeeze and exude liquid therefrom.

The present invention is particularly concerned, but not exclusively, with a urethra pressure control adjustable valve which essentially replaces the prostate in men. The prostate is a male gland about the size and shape of a walnut that surrounds the urethra immediately below the bladder. To treat prostate cancer the prostate gland is usually surgically removed and this could cause problems to the muscles that control the bladder amongst other side effects. The loss of control by the bladder muscles will cause uncontrollable leakage. Various methods and devices have been developed to try and treat this problem. One such treatment is to insert a catheter through the urethra to drain the bladder. The catheter then leads to a bag in which the fluid from the bladder is collected. A major problem with these catheters is that they often develop infections and stone formation not to mention the discomfort of carrying and empting bag on a regular basis. They also require frequent disinfecting and cleaning. Cauterization is usually done by a doctor but a patient may be easily trained to effect the procedure himself. To do this, there is a need to learn sterile techniques to avoid urinary track infections.

A more recent technique is to use an artificial sphincter which is implanted adjacent the urethra below the bladder to keep the urethra closed until it is time to urinate. As reported in medical publications, this device can help people who have incontinence because of weak sphincter muscles or because of nerve damage that interferes with sphincter muscle function. It does not solve incontinence caused by uncontrolled bladder contraction. Artificial sphincters consist of a cup that fits around the urethra with a small balloon reservoir placed in the abdomen and a pump placed in the scrotum. The cup is filled with a liquid that makes it fit tightly around the urethra to squeeze the urethra to prevent urine from leaking. When it is time to urinate you squeeze the pump with the fingers to deflate the cup so that the liquid moves to the balloon reservoir from the cuff and urine can now flow through the urethra. When the bladder is emptied, the cup automatically refills within a time delay of about 2 to 5 minutes to keep the urethra tightly closed. This solution has not been found to work efficiently and requires interaction with the user to release the urine.

In recent years a new procedure has been developed to treat urinary incontinence. This new procedure comprises implanting a balloon which is connected to a conduit tube with the conduit tube remaining inside a person's body and the balloon is positioned adjacent the urethra whereby upon inflation of the balloon, through the scrotum, the urethra will be squeezed and hopefully close. The tube is provided with an inlet port positioned in the scrotum and through which a fluid is injected by a syringe, through the skin of the scrotum, whereby to inject a proper amount of fluid in the balloon to expand it to apply sufficient pressure against the urethra. This technique has also encountered various problems, and it has been reported that the success rate is no better than fifty percent (50%). A major problem with this technique is that the urethra is unstable and when pressure is applied against it the urethra will be displaced in an uncontrollable manner. The balloons are also unstable. This is why the efficiency rate has not been satisfactory. Usually there are two of these balloons that are implanted one on opposed sides of the urethra and sometimes offset from one another. Reference to U.S. Pat. Nos. 6,045,498 and 6,445,138 describes such implantable devices and their operation.

As reported in Medical News Today, Newsletter dated Oct. 24, 2006, these balloons are implanted beneath the bladder neck to increase its resistance. The novel difference with this device is the ability to adjust the tightness of the urethral occlusion by altering the amount to fluid in each balloon via a titanium port connector that can be accessed via a percutaneous injection in the scrotum. A study of this technique is also reported in the May 2006 issue of Urology. With this technique balloon adjustment is required to achieve continence and the average number of adjustments was 4.6, all of which were done in an out patient setting and in first six months after placement. A revision surgery was also required in four of twenty-three patients.

The above-mentioned technique appears to be on course to eventually resolve problems associated with balloon implants. However, there is still a need to resolve major problems with this technique such as the assurance that the implant will effectively engage the urethra and effect proper closure thereof by applying a pressure customized to the patient's needs depending on his degree of control to evacuate urine from the bladder. Another problem to be resolved is the implantation of the device itself about the urethra to effectively assure the proper function thereof prior to closing the incision.

It has also been reported by NIDDK that women experience incontinence twice as often as men. Pregnancy and childbirth, menopause, and the structure of the female urinary tract account for this difference. But both women and men can become incontinent from neurologic injury, birth defects, strokes, multiple sclerosis and physical problems associated with aging. Incontinence in women usually occurs because of problems with muscles that help to hold or release urine.

Many types of treatments are used to treat incontinence in women, depending in the severity of their problem, such as exercises, electrical stimulation, biofeedback timed voiding or bladder training, medications, pessaries, implants, surgery and catherization.

### SUMMARY OF INVENTION

It is a feature of the present invention to provide a surgically implantable urethra pressure control adjustable valve which substantially overcomes the above-mentioned disadvantages of the prior art.

Another feature of the present invention is to provide a surgically implantable urethra pressure control adjustable valve which is easy to install and provides visibility to the surgeon when positioning the valve and its pressure control means against the urethra.

Another feature of the present invention is to provide a surgically implantable urethra pressure control adjustable valve, the closing pressure of which is adjustable through the scrotum or elsewhere by injecting fluid into a balloon retained in the valve through a conduit provided with a port connector located in the scrotum or elsewhere.

According to the above features, from a broad aspect, the present invention provides a surgically implantable urethra pressure control adjustable valve. The valve is comprised of a clamp positionable about a urethra in a patient's body. The clamp has a retention wall having a first and a second opening. The first and second openings are oppositely spaced-apart to provide for the passage of the urethra through the retention wall. Pressure abutment means is provided inside the circumferential wall and disposed adjacent opposed inner wall surfaces of the retention wall on opposed sides of the urethra. At least one of the pressure abutment means is actuable to expand to apply a contained controlled pressure against the urethra to close the urethra by pinching same between said pressure abutment means. The contained controlled pressure is adjustable and selected so that liquid pressure from a bladder associated with the urethra and under pressure by muscle control will cause the urethra to open against the contained controlled pressure to discharge liquid from the bladder and to automatically close once the pressure from the bladder is discontinued by muscle control.

### BRIEF DESCRIPTION OF DRAWINGS

A preferred embodiment of the present invention will now be described with reference to the accompanying drawings in which:

FIG. 1 is a schematic illustration of the surgically implantable urethra pressure control adjustable valve of the present invention secured to a urethra below the bladder and provided with a conduit and port connector wherein fluid is injected in the balloon of the valve by a syringe;

FIG. 2. is an enlarged perspective view of the pressure control adjustable valve of the present invention;

FIG. 3 is a side view of the pressure control adjustable valve of the present invention;

FIGS. 4A to 4C are side views of the pressure control adjustable valve of the present invention and secured to the urethra to illustrate the operation thereof;

FIG. 5 is a perspective view of a further embodiment of the pressure control adjustable valve of the present invention;

FIGS. 6A to 6D are end views of a further example of the construction of the pressure control adjustable valve and wherein the clamp is of oval-like shape with opposed expandable balloons immovably retained therein on opposed sides of a urethra locating passage;

FIG. 6E is a side view of FIGS. 6A to 6D; and

FIG. 7 is a simplified section view illustrating the construction of a pressure release probe to unblock a pinched urethra.

### DESCRIPTION OF PREFERRED EMBODIMENTS

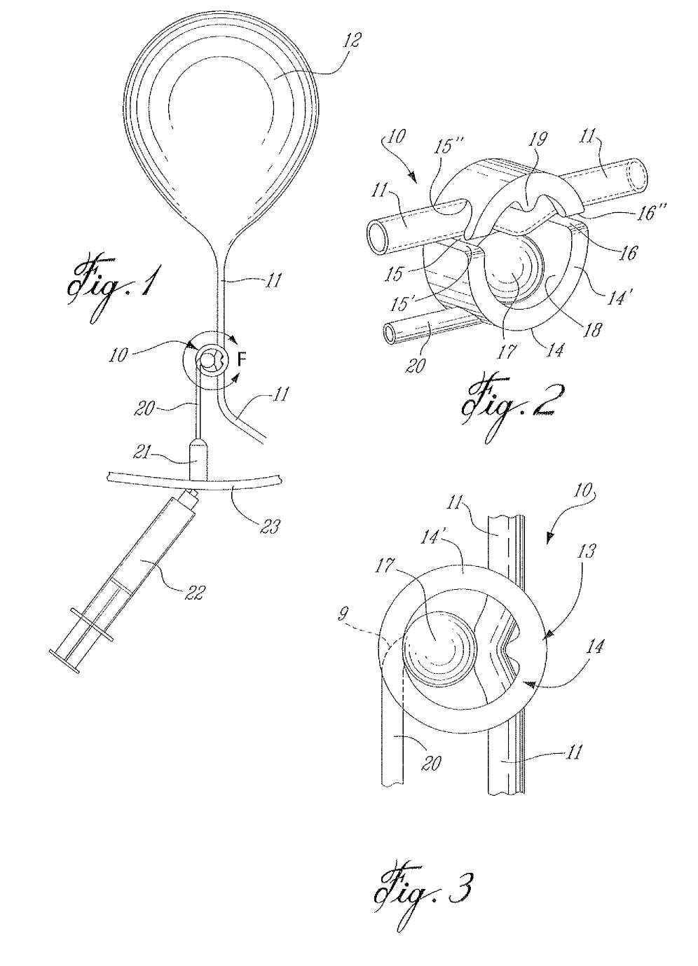

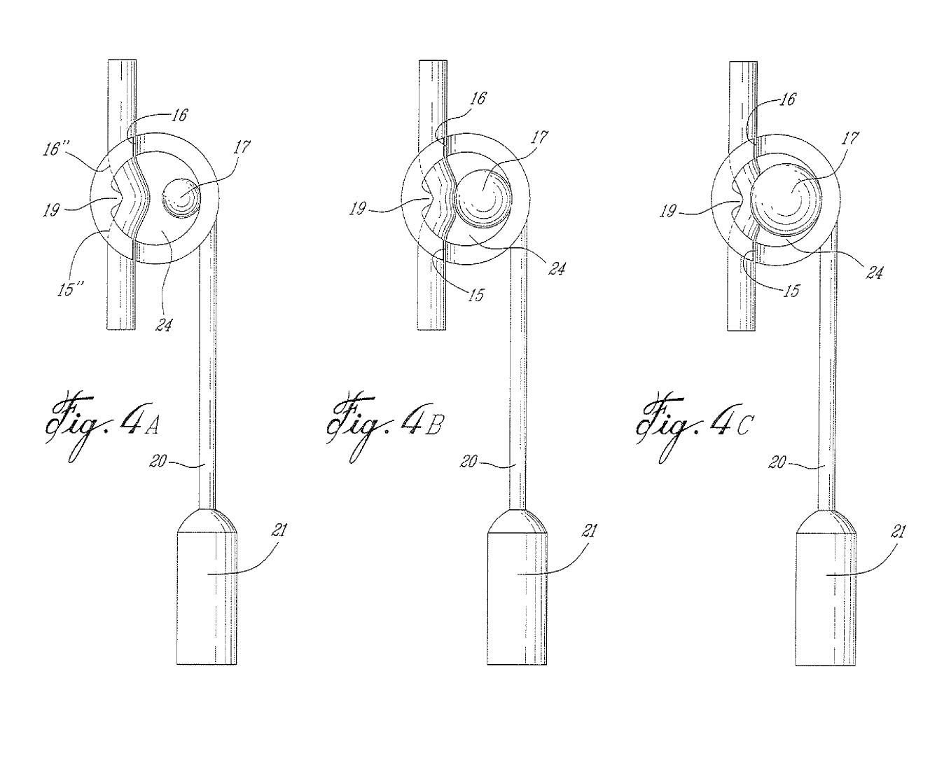

Referring now to the drawings and more particularly to FIGS. 1 to 3, there is shown generally at 10, one embodiment of the urethra pressure control adjustable valve of the present invention. As hereinshown the valve 10 is surgically implanted about the urethra 11 below the bladder 12 of a male or female patient. The valve 10 is constituted by a clamp-type design which consists of a circumferential ring 13 constructed of clinically acceptable material suitable to be implanted in a patient's body. The ring 3 defines a circumferential wall 14 which is provided with a first and second opening therein and which are oppositely spaced-apart at predetermined locations in the wall 14 to provide for the insertion and passage of the urethra 11 therethrough.

Pressure abutment means is provided in the form of a balloon 17 which is retained at a precise location inside the circumferential wall 14 by a locating through bore 9 through which the conduit of the balloon is retained. The balloon is disposed between an inner wall surface 18 of the circumferential wall 14 and the urethra 11 whereby to apply a contained control pressure against the urethra to cause the urethra to close by pinching same against a diametrically opposed abutment means, herein an immovable abutment shoulder or rib 19. This shoulder 19 is molded integral with the circumferential wall 14 and projects inwardly therein. The contained control pressure within the balloon 17 is provided by a fluid which is injected into the balloon 17 through the conduit 20. The conduit 20 is provided with a port connector 21 which is located within the scrotum against the inside surface of the outer skin 23 thereof for a male patient and under the skin at a convenient location for a female patient. A syringe 22 is used to inject the fluid through the skin 23. The port connector 21 is of a type well known in the art and prevents back leakage of the fluid. Accordingly, by controlling the amount of fluid injected in the balloon the pressure against the urethra can be controlled.

FIGS. 4A to 4C illustrate how the urethra located within the valve is closed. The proper closing pressure is selected for the patient and the selected volume of liquid is injected in the balloon to provide the selected pressure. This selected pressure will be overcome by liquid pressure from the bladder 12 associated with the urethra 11 by pressure exerted on the bladder by the sphincter muscle. This will cause the urethra to open against the selected contained control pressure in the balloon whereby to discharge liquid from the bladder. Once the bladder pressure ceases, by muscle control, the valve automatically closes. Accordingly, the operation of the device is quite simple.

The advantages of the pressure control adjustable valve design of the present invention provides many advantages over the prior art. One such advantage is that the circumferential wall 14 defines opposed open sides 24 to provide visibility to the surgeon to the first and second openings 15 and 16, and to the position of the urethra between the immovable abutment shoulder 19 and the pressure abutment balloon 17, as clearly shown in FIG. 4B. The valve design facilitates the installation of the clamp by permitting proper positioning of the clamp about the urethra. Therefore, the surgeon has complete visibility for the installation of the clamp and can visualize the action of pressurizing the balloon against the urethra prior to the closure of the incision. This assures that the balloon is in proper position to act against the urethra to pinch it to a closed position against the abutment element.

A still further important characteristic of the valve 10 of the present invention is that the first and second openings 15 and 16 are in the form of apertures in the circumferential wall 14 and are provided with a slot entrance opening in a side edge 14′ of the ring-shaped clamp. As shown more clearly in FIG. 2, the passages 15 and 16 or first and second openings are constituted as an L-shaped slot which has a first leg 15′ and 16′, respectively, which extend from the side edge 14′ and into the circumferential wall 14. A second leg section 15″ and 16″ extends substantially transverse to the first leg section and into the circumferential wall 14 whereby to define a urethra retention slot. With the balloon still inflated during operation and opening of the urethra, the urethra is captively retained in this slot and cannot slip out.

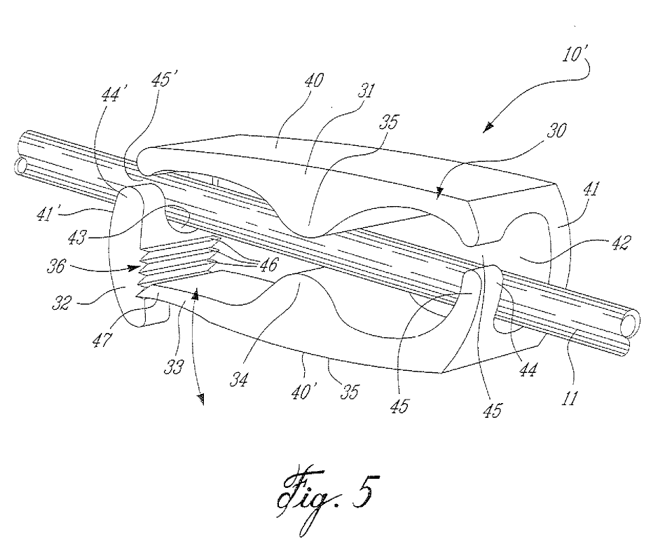

Referring now to FIG. 5, there is shown another embodiment of the construction of the clamp. As hereinshown the pressure control adjustable valve 10′ is constituted by a clamp 30 which also has a circumferential wall 31 but herein provided with opposed free ends 32 and 33 which are connectable together at a selected position by pressure connecting means which will be described later. The pressure connecting means is herein constituted by a first formation 34 which replaces the balloon and which projects inwardly of the circumferential wall 31 in a displaceable wall section 34 of the circumferential wall 31. This pressure abutment formation 34 is aligned adjacent the immovable formation 35 provided in the opposite wall of the clamp 30.

The free ends 33 and 32 of the clamp 30 are designed to interconnect together by an adjustable connector 36 whereby to adjust the distance between the ends of the formations 34 and 35 to clamp the urethra 11 therebetween. It is pointed out that the circumferential wall 31 is very thin in its portions where it is free of the formations 34 and 35 whereby to provide for a contained control pressure of the formation 34 against the urethra.

As shown in FIG. 5, the clamp 10′ is an elongated substantially rectangular shaped clamp having opposed elongated side walls 40 and 40′ and convexly curved opposed end walls 41 and 41′. The first and second openings 42 and 43 are apertures formed in the convexly curved opposed ends walls and diametrically opposed from one another. The entrance means to these apertures is provided by flexible ribs 41 and 44′ formed between the edge 45 of the clamp and the entrances 45 and 45′. Again this clamp 30 is constructed of a material, such as plastic, having a memory capable of assuming its initial shape when deformed. Accordingly, when the ribs 44 and 44′ are flexed to provide the positioning of the clamp about the urethra 11, these ribs immediately reassume their initial position. Accordingly, the urethra is held captive and aligned through the clamp and between the projecting formations 34 and 35 to assure proper position and operation of the valve.

To adjust the proper pressure, to pinch the urethra closed with a selected pressure, the adjustable connector 36 is constituted by at least two or more transverse notches 46 formed in the curved end wall 41′ and with the free end of the side wall 40′ being provided with a straight engaging end formation 47 to engage under pressure within a selected one of the notches 46. Accordingly, when this valve is installed, the proper pressure notch is selected to suit the patient as this pressure cannot be controlled from the exterior after it has been implanted. Such clamp is therefore suitable for only certain patient incontinence disorders which are more easily controllable. The adjustable connector 36 is also designed to prevent accidental disconnection.

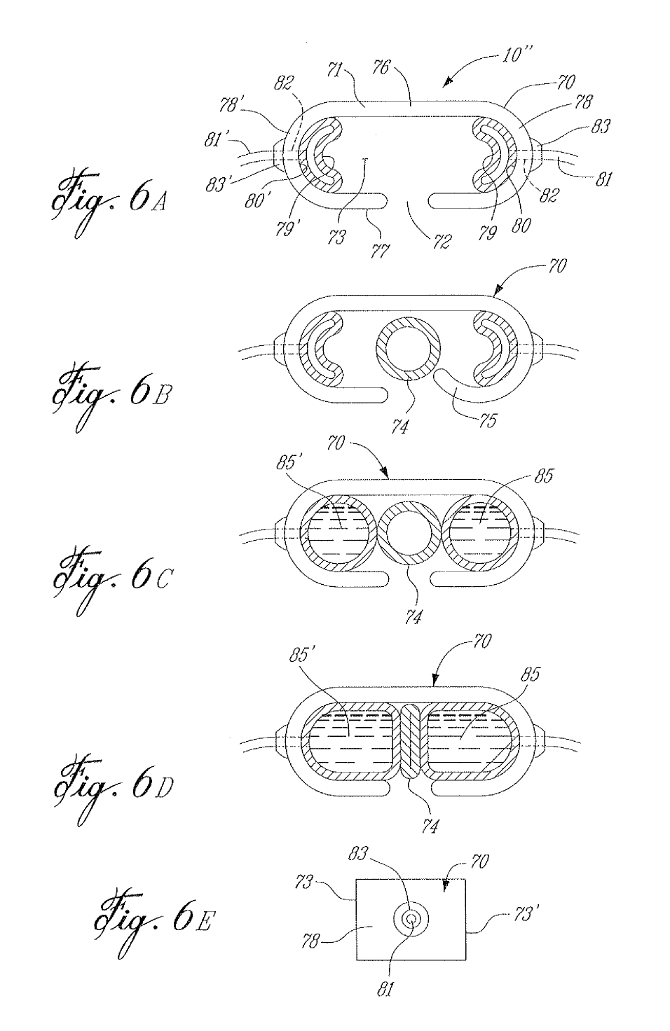

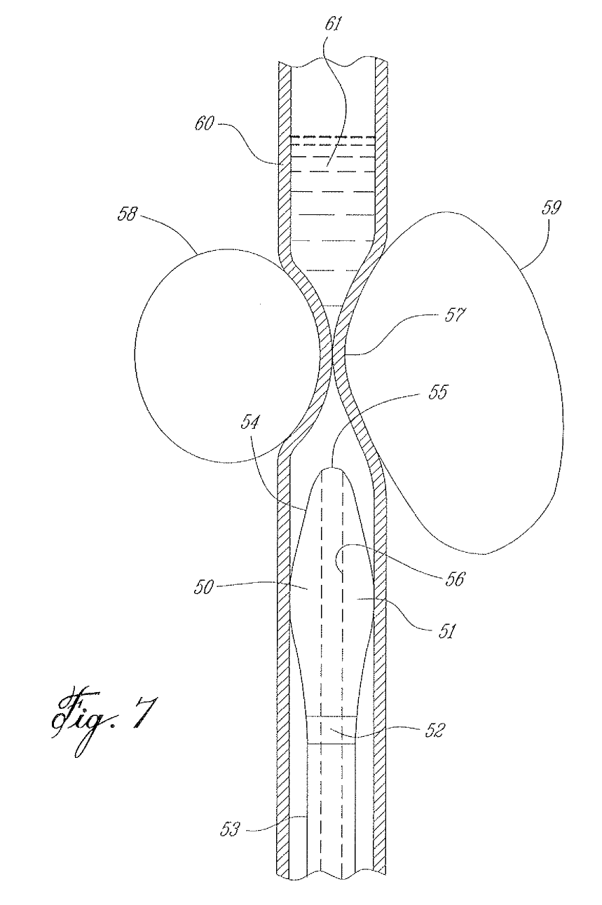

Referring now to FIGS. 6A to 6E, there is shown a further embodiment of the construction of the clamp of the present invention. As hereinshown the pressure control adjustable valve 10″ is comprised of a ring-like shaped clamp 70 with the retention wall 71 being a circumferential wall having a slot opening 72 extending across the retention wall 71 from a first side opening 73 to a second side opening 73′ as shown in FIG. 6E. The slot opening 72 permits the positioning of a urethra 74 inside and extending through the circumferential wall 71 from the first opening 73 to the second opening 73′. This permits the retention wall to be positioned about the urethra.

The clamp 70 is constructed of a flexible material suitable for implantation and wherein by flexing the wall portion 75 on either side of the slot opening, the slot opening 72 can be enlarged whereby to permit passage of the urethra therethrough. Thereafter the memory of the material forming the retention wall re-assumes its position thereby preventing the urethra from sliding out of the clamp. It is pointed out that the drawings as illustrated in FIGS. 6A to 6E are of exaggerated dimensions but in actual use this clamp is very small and a surgical tool is used whereby to bend the clamp portion adjacent the slot opening to permit its positioning about the urethra. This slot opening dimension constitutes a means to retain the urethra captive through the retention wall.

As shown in these drawings the ring-like shaped clamp 70 is an oval-shaped clamp having opposed elongated top and bottom walls 76 and 77, respectively and opposed curved side walls 78 and 78′. The slot opening 72 extends across the bottom wall 77 and substantially centrally therein.

The pressure abutment means is herein constituted by a pair of balloons 79 and 79′ retained against the inner surfaces 80 and 80′ of the curved side walls 78 and 78′, respectively, and this is accomplished by locating the conduits 81 and 81′, respectively, connected to these balloons through bores 82 and 82′ formed in these curved side walls. Connectors 83 and 83′ connect the conduits 81 and 81′, respectively, to these curved side walls. These balloons 79 and 79′ are assembled with the ring-like shaped clamps 70 during manufacture and as shown in FIG. 6A, these balloons are in an unexpanded form with no fluid present in the balloons. Accordingly, during installation there is sufficient space inside the clamp to locate the urethra 74 as shown in FIG. 6B.

Referring now to FIGS. 6E and 6D, it can be seen that once the clamp is engaged with the urethra 74, fluid 85 and 85′ are injected into the balloons 79 and 79′ whereby to close the urethra 74 as illustrated in FIG. 6D. The pressure of this fluid 85 and 85′ is selected to suit the patient's condition of incontinence, as previously described. It can therefore be seen that with this further embodiment the balloons are always retained diametrically opposed on a respective side of the urethra thereby ensuring proper operation. A particular advantage of this clamp is its simplicity in construction and assembly whereby the balloons are always retained in perfect alignment on opposed sides of the urethra.

FIG. 7 illustrates a pressure relieve catheter probe which is suitable to unblock a pinched urethra. The patient is trained to utilize this probe in a sanitary manner should a pinched urethra fail to open. This type of probe would only provide for temporary relief and one would have to consult a physician for proper remedial procedure. The probe 50 comprises a rigid shaped body 51 which is of tubular shape and which is secured at a rear end 52 thereof to a semi-rigid hollow tube 53. The probe 50 has a pointed end 54 with a smoothly rounded tip 55. An open conduit 56 extends from the rounded tip 55 to the rear end 52 of the probe and is in communication with the hollow tube 53.

As shown in FIG. 7, the urethra 60 is being pinched or blocked in the region 57 by either a balloon 58 or surrounding tissue 59 and in order to provide for relief of the fluid 61 within the bladder the probe is injected into the urethra from its free end and pushed to the pinched area 57 of the urethra to unblock it and to evacuate the liquid 61 from the bladder through the conduit 56 and the hollow tube 53. By inserting the probe 50 within the blocked or pinched region 57, this may cause the fat tissue or balloon to reposition itself in a manner to cause the pinched area to function normally and open upon liquid pressure from the bladder above the pinched area. If not, then the probe can be inserted again and a physician consulted for corrective action.

It is within the ambit of the present invention to cover any obvious modifications of the examples of the preferred embodiment described herein provided such modifications fall within the scope of the appended claims.

I claim:

1. A surgically implantable urethra pressure control adjustable valve comprising a clamp for positioning about a urethra in a patient's body, said clamp having a retention wall, said retention wall having a first and a second opening, said first and second passage openings being oppositely spaced-apart to provide for the passage of said urethra through said retention wall, pressure abutment means inside said circumferential wall and disposed adjacent opposed inner wall surfaces of said retention wall on opposed sides of said urethra, at least one of said pressure abutment means being actuable to expand to apply a contained controlled pressure against said urethra to close said urethra by pinching same between said pressure abutment means, said contained controlled pressure being adjustable and selected so that liquid pressure from a bladder associated with said urethra and under pressure by muscle control will cause said urethra to open against said contained controlled pressure to discharge liquid from said bladder and to automatically close once said pressure from said bladder is discontinued by said muscle control.

2. A surgically implantable urethra pressure control valve as claimed in claim 1 wherein said retention wall openings provide visibility to a surgeon implanting said valve, said pressure abutment means being unexpanded to facilitate the installation of said clamp and the positioning of said urethra therethrough.

3. A surgically implantable urethra pressure control valve as claimed in claim 2 wherein said clamp is a ring-shaped clamp with said retention wall being a circumferential retention wall, said first and second passage openings being substantially diametrically opposed from one another and entrance means to said passage openings to position said openings about said urethra.

4. A surgically implantable urethra pressure control valve as claimed in claim 3 wherein one of said pressure abutment means is an expandable abutment means, the other abutment means being a diametrically opposed immovable abutment means projection formed in said inner wall surface.

5. A surgically implantable urethra pressure control valve as claimed in claim 4 wherein said entrance means is a slot entrance opening in a side edge of said clamp, and a flexible rib formed by said clamp between said edge and said passage, said flexible rib being flexed to permit said passage to be expanded to permit the insertion of said urethra within said passages, said clamp being constructed of material having a memory capable of assuming its initial shape when deformed.

6. A surgically implantable urethra pressure control valve as claimed in claim 4 wherein said passage openings are each constituted by an L-shaped slot having a first leg section extending from a side edge of said ring-shaped clamp into said circumferential wall, and a second leg section extending substantially transverse to said first leg section into said circumferential wall to define a urethra retention slot, said urethra being captively retained in said urethra retention slot.

7. A surgically implantable urethra pressure control valve as claimed in claim 4 wherein said ring-shaped clamp is further provided with a retention through bore in said circumferential wall, said retention through bore being diametrically aligned and opposite said immovable abutment means, said retention through bore retaining said pressure abutment means inside said circumferential wall.

8. A surgically implantable urethra pressure control valve as claimed in claim 7 wherein said pressure abutment means is a balloon connected to a conduit, said conduit extending through said retention through bore to retain said balloon opposite said immovable abutment means.

9. A surgically implantable urethra pressure control valve as claimed in claim 4 wherein said ring-shaped clamp is formed of a clinically acceptable material suitable to be implanted in a patient's body.

10. A surgically implantable urethra pressure control valve as claimed in claim 4 wherein said pressure abutment means is provided by an expandable element capable of retaining a fluid therein under pressure, a conduit connected to an interior of said expandable element to provide for the insertion of a pressurized fluid therein, said conduit having a self-sealing port connector at a free end thereof to receive a fluid injected therethrough in sealing arrangement, said port connector automatically sealing itself upon retraction of said needle.

11. A surgically implantable urethra pressure control valve as claimed in claim 10 wherein said fluid is a liquid.

12. A surgically implantable urethra pressure control valve as claimed in claim 10 wherein said free end of said conduit is placed in the scrotum of a male patient at a locatable position whereby said needle can be positioned in said port connector through an outer skin of said scrotum.

13. A surgically implantable urethra pressure control valve as claimed in claim 10 wherein said immovable abutment means is a smoothly curved ridge formation integrally formed and transversely extending across said circumferential wall and disposed transversely of a straight axis passing through said first and second openings.

14. A surgically implantable urethra pressure control valve as claimed in claim 1 wherein said circumferential wall has opposed free ends secured together by an adjustable connector, said pressure abutment means being constituted by a first formation projecting inwardly in said circumferential wall in a displaceable wall section thereof adjacent said adjustable connector and a second formation projecting inwardly in said circumferential wall and diametrically aligned with said first formation, said adjustable connector adjusting the distance between clamping free ends of said first and second formations to clamp said urethra therebetween, said circumferential wall being a thin flexible wall housing having a predetermined memory to provide said contained controlled pressure and to flex under the influence of said liquid pressure from said bladder under the influence of sphincter muscle control.

15. A surgically implantable urethra pressure control valve as claimed in claim 14 wherein said free end of said circumferential wall adjacent said first formation has a straight engaging end formation, said other free end being provided with at least two transverse notches to immovably retain said straight engaging end formation in pressure contact therewith, said transverse notches providing for selective clamping pressure of said urethra.

16. A surgically implantable urethra pressure control valve as claimed in claim 15 wherein said clamp is an elongated rectangular clamp having opposed elongated side walls and convexly curved opposed end wall, said first and second passage openings being formed in said convexly curved opposed end walls and diametrically opposed from one another, and entrance means to said openings to position said openings about said urethra.

17. A surgically implantable urethra pressure control valve as claimed in claim 16 wherein said entrance means is a slot entrance opening in a side edge of said clamp, and a flexible rib formed by said clamp between said edge and said passage, said flexible rib being flexed to permit said passage to be expanded to permit the insertion of said urethra within said passages, said clamp being constructed of material having a memory capable of assuming its initial shape when deformed.

18. A surgically implantable urethra pressure control valve as claimed in claim 16 wherein said first and second formations are smoothly curved ridge formations integrally formed in a respective one of said opposed elongated side walls.

19. A surgically implantable urethra pressure control valve as claimed in claim 16 wherein said circumferential wall defines opposed open sides to provide visibility to said first and second openings, said immovable abutment means and said pressure abutment means to facilitate the installation of said clamp and the positioning of said urethra therethrough.

20. The use of the surgical implantable urethra pressure control adjustable pressure valve as claimed in claim 1 to control male and female incontinence.

21. The use of the surgical implantable urethra pressure control adjustable pressure valve as claimed in claim 1 wherein said clamp is a ring-like shaped clamp with said retention wall being a circumferential wall having a slot opening extending thereacross from said first and second opening to permit the positioning of said urethra inside and through said circumferential wall and extending from said first and second opening whereby said retention wall is positionable about said urethra, and means to retain said urethra captive through said retention wall.

22. The use of the surgical implantable urethra pressure control adjustable pressure valve as claimed in claim 21 wherein said slot is dimensioned to retain said urethra captive in said retention wall, at least a side wall portion of said circumferential wall adjacent said slot being flexible to permit expansion of said slot for insertion of said urethra therethrough, said slot dimension constituting said retention means.

23. The use of the surgical implantable urethra pressure control adjustable pressure valve as claimed in claim 21 wherein said pressure abutment means is an expandable abutment means immovably secured to a respective one of said adjacent opposed inner wall surfaces on a respective side of said slot opening.

24. The use of the surgical implantable urethra pressure control adjustable pressure valve as claimed in claim 23 wherein said ring-like shaped clamp is an oval shaped clamp having opposed elongated top and bottom walls and opposed curved side walls, said slot extending across one of said top and bottom walls substantially centrally therein, said expandable abutment means being secured to said curved side walls.

25. The use of the surgical implantable urethra pressure control adjustable pressure valve as claimed in claim 24 wherein said expandable abutment means is provided by an expandable element capable of retaining a fluid therein under pressure, a conduit connected to an interior of said expandable element to provide for the insertion of a pressurized fluid therein, said conduit having a self-sealing port connector at a free end thereof to receive a fluid injected therethrough in sealing arrangement, said port connector automatically sealing itself upon retraction of said needle.

26. The use of the surgical implantable urethra pressure control adjustable pressure valve as claimed in claim 25 wherein said expandable element is retained captive by said conduit being retained captive in a through bore extending in said opposed curved side wall, and a connector to connect said conduit to said curved side wall whereby to retain said expandable element adjacent said inner wall surfaces.

27. A pressure relieve probe to unblock a pinched urethra, said probe having a cylindrical rigid shaped body secured at a rear end thereof to a semi-rigid hollow tube to displace said probe in said urethra, said probe having a pointed end with a smoothly rounded tip, and an open conduit extending from said rounded tip to said rear end in communication with said hollow tube.

28. A pressure relieve probe as claimed in claim 27 wherein said open conduit has a diameter sufficient to permit the flow of urine therethrough.
